(19) United States
(12) Patent Application Publication
(10) Pub. No.: US 2025/0292913 A1
(43) Pub. Date: Sep. 18, 2025

(54) METHOD FOR SETTING UP AN ORTHOPAEDIC DEVICE

(71) Applicant: Ottobock SE & Co. KGaA, Duderstadt (DE)

(72) Inventors: Andreas Bohland, Vienna (AT); Christian Daur, Berlin (DE); Alexander Glier, Duderstadt (DE); Dries Glorieux, Lommel (BE); Sven Zarling, Duderstadt (DE); Erik Albrecht-Laatsch, Rosdorf (DE); Mark Schönemeier, Göttingen (DE); Roland Auberger, Vienna (AT); Marcus Eder, Vienna (AT)

(21) Appl. No.: 18/862,402

(22) PCT Filed: May 5, 2023
(86) PCT No.: PCT/EP2023/061967

§ 371 Date: Nov. 1, 2024

(30) Foreign Application Priority Data
May 6, 2022 (DE) 10 2022 111 332.9

(51) Int. Cl. G16H 50/50; A61F 2/60; A61F 2/76; G06T 11/00; G16H 20/30; G16H 40/67

(52) U.S. Cl. CPC G16H 50/50; A61F 2/60; G06T 11/00; G16H 20/30; G16H 40/67; A61F 2002/769; G06T 2210/41

(57) ABSTRACT

The invention relates to a method for the computer-based setting up of an orthopaedic device which is worn on the body of a patient provided therewith, the method comprising the following steps: —recording status-related and/or movement-related data from the orthopaedic device during a movement sequence in which the patient in question performs a movement with the orthopaedic device, —transmitting the recorded status-related and/or movement-related data relating to the movement sequence to an augmented-reality device which is being used by a user, and —using the augmented-reality device to overlay at least some of the transmitted status-related and/or movement-related data and/or information derived therefrom, in the form of augmented reality, onto the reality perceived by the user.

The invention relates to a method of computer-based configuration of an orthopedic apparatus worn on the body of a patient equipped therewith. The invention also relates to a system to this end.

Within the meaning of the present invention, orthopedic apparatuses are in particular orthoses, prostheses, exoskeletons and optionally also wheelchairs. Orthoses are products that support, assist, protect or restrict the freedom of movement of a body part of the patient, for example a joint, in order to avoid overuse. By contrast, prostheses replace body parts of the patient that are not, or no longer, present. Exoskeletons are mechanical support structures in particular, which are intended to support, assist or protect the main musculoskeletal system of the patient.

Hereinbelow, a patient is understood to mean any user of the orthopedic apparatus. This consequently relates to the wearer of the orthopedic apparatus.

Every orthopedic apparatus is arranged on a body part of the patient. There need not necessarily be contact with the skin of the patient. For example, orthoses and exoskeletons are frequently worn above the apparel, and so the latter, for example a pair of trousers, is situated between the orthopedic apparatus and the skin of the patient. Nevertheless, a knee orthosis is secured to the knee or the leg of the patient, for example. A prosthesis always comprises an interface element that is connected to an amputation stump or any other body part and secured to the respective body part. In the case of a leg prosthesis, use is made of a prosthesis socket, for example, which represents the interface between prosthesis and amputation stump. In this case, the amputation stump would be the body part of the patient. As a rule, a liner is used between the skin surface of the amputation stump of the patient and the prostheses socket in order to reduce shearing forces that act on the skin.

As a rule, a prosthesis socket for an amputation stump is produced from rigid (hardly deformable) material, for example a fiber-reinforced plastic, and forms an important part of the interface between the amputation stump and the prosthesis arranged on the prosthesis socket. Appropriate prosthesis sockets have been used for a long time, especially for leg prostheses that should be arranged on an amputation stump, for example a thigh.

Prosthesis sockets for leg amputees, in particular, are exposed to particular loads during daily use. When walking, the entire weight of the patient bears on the prosthesis socket, and hence in particular on the amputation stump arranged in the prosthesis socket. It is therefore very important to adapt the prosthesis socket as optimally as possible to the individual circumstances and needs of the patient, in particular to the shape and geometry of the relevant body part.

The patient is assisted by a technologically educated user, for example an orthopedic technician, when fitting and configuring an orthopedic apparatus. The orthopedic technician can draw on their knowledge and experience to implement the necessary adjustments so that the orthopedic apparatus fits optimally and a sparing movement pattern arises.

EP 2 153 370 B1 has disclosed a system for aligning prostheses, in which both movement data of the person equipped with the prostheses and prostheses alignment errors are ascertained from a movement database. These are compared with one another in order to determine whether the prostheses corresponds to a target value or requires further adjustments.

DE 10 2012 009 507 A1 has disclosed a method and an apparatus for determining incorrect positions in the construction of prostheses of the lower extremities, wherein inertial measurement data are ascertained over at least one gait cycle and are compared to target values.

DE 10 2018 128 514 B4 has disclosed a method for carrying out static prostheses construction for a prosthesis, wherein a plurality of components are arranged on one another. In this case, an actual position and an actual orientation of the components arranged on one another are ascertained relative to one another on the basis of detected positions and orientations of markers and are compared to appropriate target values.

Against this background, the problem addressed by the present invention is that of proposing an improved method for configuring an orthopedic apparatus.

According to the invention, the problem is solved by the method as claimed in claim 1.

Advantageous embodiments of the invention are found in the corresponding dependent claims.

According to claim 1, a method of computer-based configuration of an orthopedic apparatus worn on the body of a patient equipped therewith is proposed, wherein the method comprises the following steps:
acquiring status-related and/or movement-related data of the orthopedic apparatus during a movement sequence, within the scope of which the relevant patient carries out a movement using the orthopedic apparatus,
transferring the acquired status-related and/or movement-related data regarding the movement sequence to an augmented-reality device used by the user, and
superimposing, in the form of augmented reality, at least some of the transferred status-related and/or movement-related data and/or information derived therefrom into the reality perceived by the user using the augmented-reality device.

In this context, configuring an orthopedic apparatus is understood to mean not only the adjustment of the settable parameters of the apparatus but also the selection and/or replacement of components and the arrangement thereof on the body of the patient and/or the alignment of various components to one another. The resultant treatment should be assessed with the aid of the present method, and this treatment should then be modified if required.

According to the invention, status-related and/or movement-related data of the orthopedic apparatus are initially acquired during a movement sequence. Acquisition of the status-related and/or movement-related data of the orthopedic apparatus can be implemented with the aid of sensors or sensor systems arranged on the orthopedic apparatus, integrated in the orthopedic apparatus and/or provided outside of the orthopedic apparatus. This can relate to sensors that are contained per se in the orthopedic apparatus as standard and that also remain there during normal use by the patient. As a rule, the movement-related data are those pieces of information that arise directly from the movement of the patient with the orthopedic apparatus and are ascertainable as physical parameters by means of sensors. For example, such parameters could be ascertained joint angles, acting forces, torques, speeds and/or movement trajectories. As a rule, the status-related data are fixed settings that are set on the orthopedic apparatus (and as a rule remain unchanged even during the movement or whose change follows fixed rules). For example, such status-related data can be damping settings of prostheses with a knee and/or ankle joint, wherein a damping profile can be specified over the gait cycle, for example. By preference, these status-related data also contain information regarding the state of the prosthesis controller, for example which mode of many is currently adopted, for example climbing stairs, bicycling or walking on flat ground, which part of the gait cycle was just recognized, whether the apparatus is under load and the extent of any load. For example, the damping level changes during the gait cycle, wherein a different profile for damping over the gait cycle can be chosen for different modes such as running or walking or ascending a ramp, descending a ramp; this can also be referred to as status-related data.

In this case, sensors can be cameras and/or depth sensors, for example, which ascertain corresponding status-related and/or movement-related data of the orthopedic apparatus with the aid of image evaluation. However, inertial sensors are also conceivable; these acquire acceleration values in at least 3 spatial directions, whereby information regarding force vectors and movement directions can be ascertained. However, force sensors arranged within the orthopedic device or present outside of the device, for example a force plate, are also conceivable. However, sensors provided for measuring angles of foldable apparatuses or for measuring other physical parameters are also conceivable.

These captured status-related and/or movement-related data of the movement sequence are now transferred to an augmented-reality device that is used by the user, for example an orthopedic technician or another specialist. The augmented-reality device is now used to superimpose, in the form of augmented reality, the previously acquired and transferred status-related and/or movement-related data and/or information derived therefrom into the reality perceived by the user using the augmented-reality device.

The present invention thus allows the user, in particular the orthopedic technician, to be put into a position where the required data and information of the orthopedic apparatus can be immediately superimposed into the actual field of vision during the adaptation or construction of an orthopedic apparatus, with the result that the user can immediately derive appropriate measures for setting the orthopedic device. Hence, the orthopedic technician is put into the position of being able to see the status-related and/or movement-related data of the orthopedic apparatus acquired during the movement sequence while the patient with the orthopedic device in question is still in motion, and hence the orthopedic technician is able to immediately correlate the movement sequence data with the reality-based perception of the movement sequence. By preference, the user can also change setting parameters of the orthopedic apparatus by way of the augmented-reality devices, and hence directly implement the conclusions drawn. For example, this can be implemented with the aid of the acoustic input (e.g. voice commands).

According to an embodiment, provision is made for at least some of the transferred status-related and/or movement-related data and/or information derived therefrom to be superimposed, in the form of augmented reality, during the movement sequence into the reality of the movement sequence perceived by the user using the augmented-reality device.

In the ideal case, the orthopedic technician consequently identifies the acquired movement-related data during the movement by the patient and identifies the status-related data in the form of settings of the orthopedic device, without this requiring said orthopedic technician to avert their gaze from the movement of the patient in order to obtain the information. Instead, the status-related and/or movement-related data are superimposed directly into the observation of the movement and thus allow a best-possible movement analysis of the orthopedic device. In particular, an advantage thereof is that the orthopedic technician can interact with the patient during the movement sequence, as is customary, and can provide said patient with instructions, for example.

According to an embodiment, provision is made for a camera connected to the augmented-reality device to be used to record the movement sequence of the movement performed by the relevant patient with the orthopedic apparatus, and for at least one positionally accurate representation position to be ascertained on the basis of the recorded movement sequence, the data and/or information correlating with the said representation position, wherein the transferred status-related and/or movement-related data and/or the information derived therefrom are superimposed, in the form of augmented reality and in positionally accurate fashion, into the reality perceived by the user at the ascertained position.

In this case, the relative alignment of the augmented-reality device is ascertained in relation to the movement sequence of the relevant patient in order thus to superimpose the respective status-related and/or movement-related data for the user into reality at the location to which the status-related and/or movement-related data with respect to the orthopedic apparatus relate.

According to an embodiment, provision is made for a movement sequence stored in a data memory and recorded previously, or a part thereof, to be provided, wherein the augmented-reality device is used to superimpose, in the form of augmented reality, the stored and previously recorded movement sequence, or a part thereof, into the reality perceived by the user.

As a result, the orthopedic technician or user is able to carry out a comparison between previous movement sequences and the current movement sequence perceived in reality, and thus compare the implemented adaptations and settings with the previous movement sequences and study the effect thereof. In this case, the stored movement sequence can be a stored, optimal movement sequence or else a movement sequence of the same patient recorded previously, for instance with other settings.

According to an embodiment, provision is made for the data and/or information to be presented on a display device worn in front of the eyes of the user, in such a way that the data and/or information are superimposed, in the form of augmented reality, into the reality perceived by the user.

In this case, reality is detected directly by the visual sensory organs of the user, and the necessary data and/or information are superimposed with the aid of the augmented-reality device into this reality perceived by the visual sensory organs, and hence the perceived reality is augmented. For example, this can be implemented with the aid of projection glasses, in which the data are projected or displayed on a lens surface of a pair of glasses.

According to an embodiment, provision is made for a camera connected to the augmented-reality device to be used to record the movement sequence of the movement performed by the relevant patient with the orthopedic apparatus and play back the said movement sequence in real time on a display of the augmented-reality device, wherein the data and/or information in the representation of the recorded movement sequence played back on the display are superimposed, in the form of augmented reality, into the reality perceived by the user.

In this case, the reality is recorded by a camera and presented one-to-one on a display such that the user can perceive the reality presented on the display using their visual sensory organs. Now, the augmentation to reality resulting from the additional data and/or information is superimposed into this presentation such that the originally recorded and presented reality is augmented by the data and information.

According to an embodiment, provision is made for the status-related and/or movement-related data to be acquired from a sensor source external to the orthopedic apparatus, from a sensor source integrated in the orthopedic apparatus and/or from a patient-related sensor source secured to the patient.

According to an embodiment, provision is made for a camera connected to the augmented-reality device to be used to record the movement sequence of the movement performed by the relevant patient with the orthopedic apparatus, and for an evaluation unit to be used to ascertain movement-related data and/or information derived therefrom from the recorded movement sequence by way of image recognition carried out by the evaluation unit, the said movement-related data and/or information derived therefrom then being superimposed in the form of augmented reality.

In this case, a detection device is used to record the movement sequence of the patient with the orthopedic device, and appropriate data and/or information are derived therefrom such that the camera forms a type of sensor system together with the detection device. The movement-related data obtained from the image recognition and/or information derived therefrom are then superimposed, in the form of augmented reality, into the detected reality of the user with the aid of the augmented reality device.

According to an embodiment, provision is made for setting parameters of the orthopedic apparatus, as status-related data and/or information derived therefrom, to be transferred to the augmented-reality device or to be already stored therein, and for the said setting parameters to be superimposed, in the form of augmented reality, into the reality perceived by the user.

According to an embodiment, provision is made for targets in relation to status-related and/or movement-related data and/or information derived therefrom to be provided in a database, wherein at least some of the targets are read from the database and are superimposed, in the form of augmented reality, into the reality perceived by the user.

As a result, not only the acquired status-related and/or movement-related data and/or information derived therefrom as actual values but also the related targets as target values can thus be displayed to the orthopedic technician or user in the augmented reality, whereby a quick comparison is rendered possible. By preference, a deviation of the attained actual values from the targets is furthermore superimposed.

According to an embodiment, provision is made for a target movement sequence to be provided in a database, wherein at least some of the target movement sequence is read from the database and superimposed, in the form of augmented reality, into the reality perceived by the user.

Thus, the target movement sequence is a target movement sequence, wherein such a target movement sequence or a part thereof is presented, in the form of augmented reality, to the user in the perceived reality, with the result that, within the scope of movement analysis, the user can carry out a comparison between the presented target movement sequence and the actual, real movement sequence.

According to an embodiment, provision in this respect is made for deviations from the target movement sequence to be determined on the basis of the status-related and/or movement-related data and/or information derived therefrom using an evaluation unit and to be superimposed, in the form of augmented reality, into the reality perceived by the user.

Such deviations from the target movement sequence, i.e. deviations of the actual values from the specified target values, can be visualized and superimposed, in the form of augmented reality, into the perceived reality. In this context, it is conceivable that the evaluation device is configured to compare the acquired status-related and/or movement-related data with the stored target movement sequence and thereby determine corresponding deviations.

According to an embodiment, provision is made for a camera connected to the augmented-reality device to be used to record the movement sequence of the movement performed by the relevant patient with the orthopedic apparatus, and for, by means of an evaluation unit, a virtual twin to be created and stored in a data memory.

In particular, such a virtual twin contains the orthopedic apparatus and the movement sequence of the patient carried out therewith and allows the representation of the movement sequence of the orthopedic apparatus and of the patient from different perspectives in order thus to be able to carry out a best-possible and comprehensive movement analysis.

Thus, an embodiment provides for recording the movement sequence to be followed by superimposing the virtual twin, in the form of augmented reality, into the field of vision of the user.

According to an embodiment, provision is also made for a modified movement sequence of the virtual twin to be calculated on the basis of a change in the settings of the orthopedic apparatus and/or by a simulation of external influences on the movement sequence by means of a computing unit, wherein the modified movement sequence is superimposed, in the form of augmented reality, into the field of vision of the user.

This makes it possible to visually detect the effects of a change in the setting values of the orthopedic apparatus directly in the perceived reality.

According to an embodiment, provision is also made for a deviation between the acquired status-related and/or movement-related data of the orthopedic apparatus and the information derived from the virtual twin to be ascertained by means of the evaluation unit and be superimposed, in the form of augmented reality, into the field of vision of the user.

Thus, it is conceivable that, with the aid of an image recognition unit, the evaluation unit recognizes movement-related data of the orthopedic device from the recorded virtual twin and subsequently compares these with movement-related data of the orthopedic apparatus acquired in sensor-assisted fashion. The result of this comparison, in particular a correspondingly recognized deviation, is then superimposed, in the form of augmented reality, into the reality of the user, and so it is possible to determine whether the corresponding sensors of the orthopedic apparatus are still working correctly.

Furthermore, the problem is also solved according to the invention by the system for configuring the orthopedic device as claimed in claim 17, wherein the system is configured to carry out the method as described above. In particular, the system comprises an augmented-reality device which, in particular, can be connected to a camera or comprises a by the camera. Moreover, an evaluation unit is provided, the latter for example can be controlled by microprocessors or microcontrollers and is configured accordingly to carry out the previously described method.

With reference to the attached figures, the invention is explained in detail and in exemplary fashion. In the drawing:

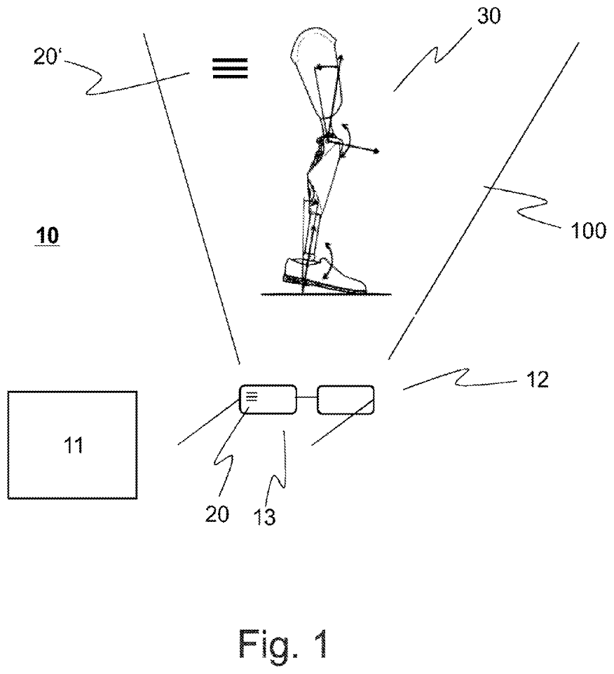

FIG. 1 shows a schematic illustration of the apparatus for carrying out the method;

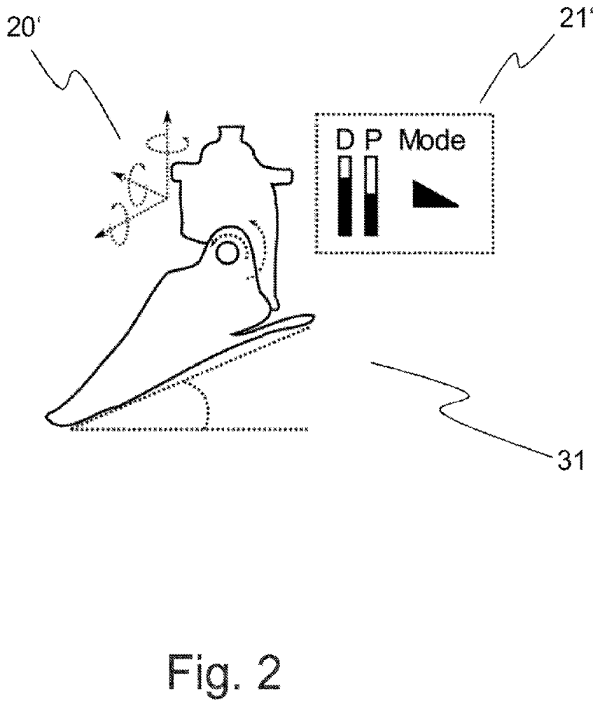

FIG. 2 shows an illustration of a prosthetic foot with additional information;

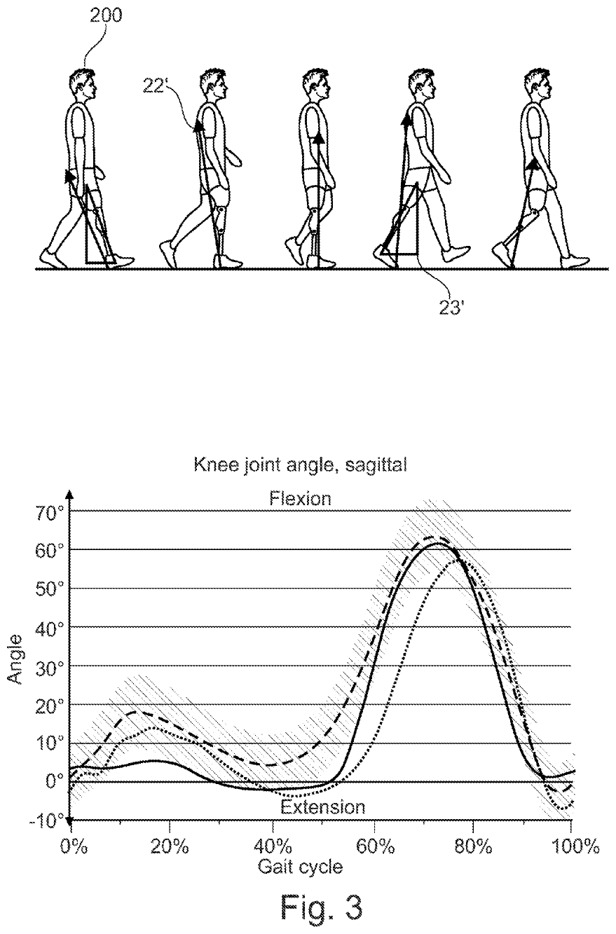

FIG. 3 shows an illustration of a gait analysis;

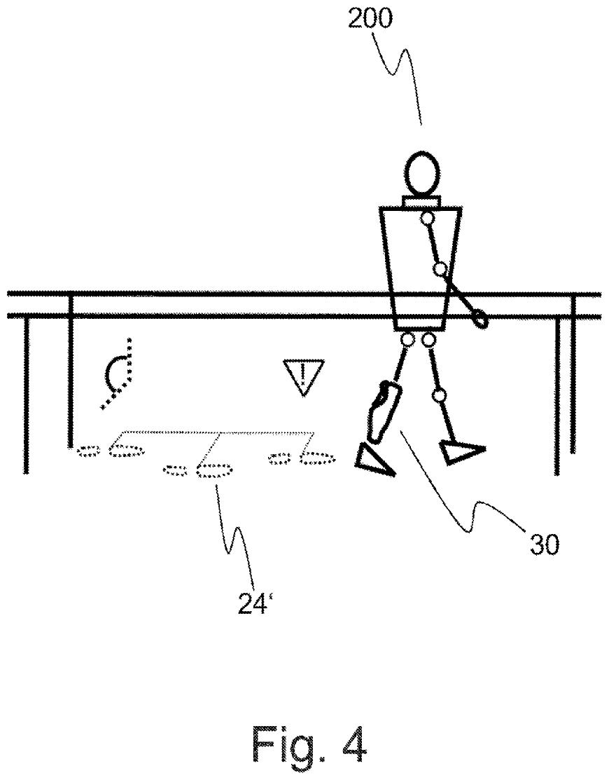

FIG. 4 shows a schematic illustration of further representations of information;

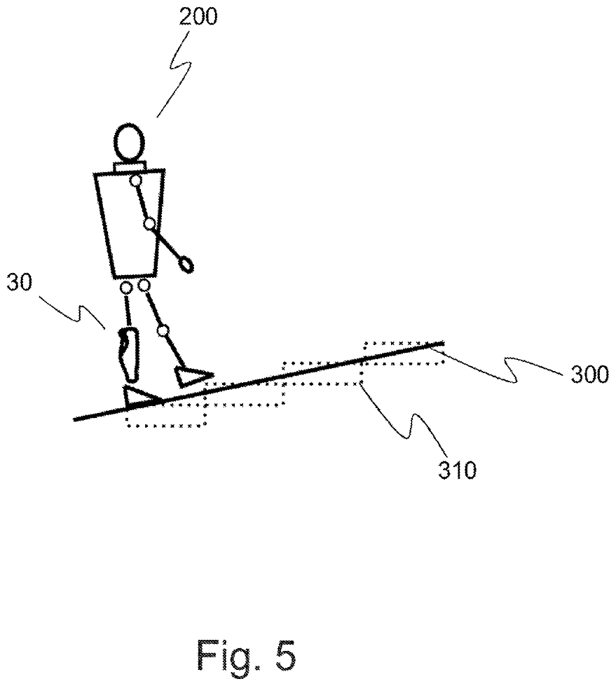

FIG. 5 shows a schematic illustration of a misconfiguration; and

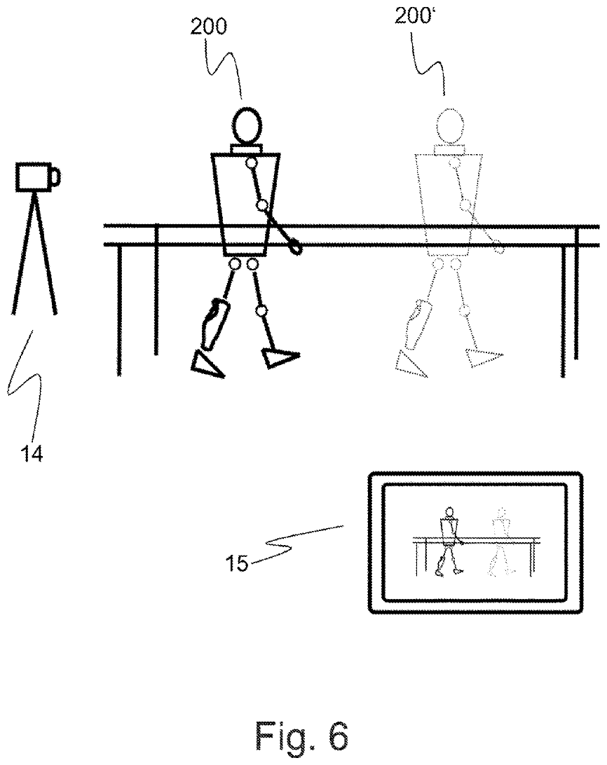

FIG. 6 shows a schematic illustration of a superimposed movement sequence.

In a schematically much simplified illustration, FIG. 1 shows an augmented-reality device 10 which in the exemplary embodiment of FIG. 1 has a computing unit or evaluation unit 11 and a projection device 12. In this case, the computing unit or evaluation unit 11 is designed such that appropriate programs, for example image recognition programs, can be executed depending on the application. In this case, the computing unit 11 is a microprocessor-controlled or microcontroller-controlled computing unit in particular.

In the exemplary embodiment of FIG. 1, the projection device 12 is depicted in the form of a pair of glasses, but it can also adopt other technical forms, for example a mobile smartphone with camera and display. In this case, the projection device 12 comprises two lenses 13 in the form of a pair of glasses, which are held in front of the eyes of the user or orthopedic technician when the glasses are worn. Then, appropriate data or information 20 can be projected or displayed on or in the lenses 13, to be precise in such a way that, in the field of vision 100, these data/this information 20 presented on the lenses 13 are/is perceivable in the field of vision 100 as data/information 20′.

In the exemplary embodiment of FIG. 1, the field of vision 100 contains a prosthesis 30 with socket, knee joint and prosthetic foot. In this case, status-related and/or movement-related data are acquired from the prosthesis 30 and transmitted to the computing unit 11. Furthermore, it is conceivable that other, external sensors are provided, which capture corresponding movement-related data and transfer these to the computing unit 11. For example, such sensors can be a force plate, on which the prosthesis 30 rolls.

Various data/items of information 20, 20′ can now be superimposed into the field of vision 100 by means of the augmented-reality device 10, the said data/items of information being recorded during the movement and being provided by the sensors of the prosthesis itself or by external sensors. For example, such parameters might be the relative angle between the lower leg in space and knee angle, axial forces, ankle moments, data from the IMU (orientation and trajectories in space), knee moments and/or knee angles.

The orthopedic technician using the augmented-reality device 10 consequently has visual contact during the movement of the prosthesis 30 on the one hand and, at the same time, has visual contact within their field of vision of the 100 with the acquired data/information 20′. In this image, arrows are used to superimpose various measurement values, in this case ankle and knee angles, floor reaction forces, lower-leg angle and thigh angle by way of example.

FIG. 2 schematically shows an illustration as possibly perceived by the orthopedic technician when wearing the projection device 12 of FIG. 1. Depicted is a mechatronic prosthetic foot 31, for which additional information is superimposed, for example the ground inclination (both the real inclination and that determined by the foot), ankle moment, ankle angle and, depicted top left, IMU data (trajectory and orientation in space). Moreover, status information 21′ for the prosthesis 30 (FIG. 1) are additionally depicted top right. Superimposed here by way of example are the setting values for the dorsal extension damping D and the plantarflexion damping P and also the current mode, which in this case symbolically represents the “descending ramp” mode.

FIG. 3 schematically shows the representation of a gait cycle of a patient 200 with a prosthetic knee and prosthetic foot. In this case, the force vector 22′ is superimposed and the position 23′ of the lower leg in space is emphasized at selected times with the aid of the augmented-reality device. In this case, the information is superimposed spatially accurately at the position of the patient 200 while the latter moves and performs a corresponding movement cycle with their prosthesis.

If need be, this information can however also remain stationary in space, even if the patient 200 has already walked on or completed the movement.

In an alternative, further information can also be depicted, for example the progression of the knee angle over the entire gait cycle. In this case, deviations from already available data can also be determined, and so the gait can be assessed by the user.

In FIG. 4, a patient 200 is depicted with a prosthesis 30 having a prosthetic foot and a prosthetic knee. In this case, the patient 200 moves forward and is stabilized by 2 hand rails.

With the aid of a camera (not depicted here), the points of set down 24′ can be detected as movement-related data and superimposed into the field of vision of the orthopedic technician, to be precise positionally accurately at the site at which the points of set down 24′ are actually located. For example, from this it is possible to calculate and likewise superimpose the step length in real time, and also, top left, the maximally attained knee flexion angle.

In a manner indicated by an appropriate symbol, it is moreover possible at a subsequent time to visualize the onset of a special mode of the prosthesis 30, for example trip identification.

In this case, the data can originate from the prosthesis itself, can be determined by the augmented-reality device itself (for example the step length) or can originate from external sensors.

FIG. 5 shows an exemplary embodiment, in which the patient 200 runs up a ramp 300 within the field of vision of the orthopedic technician. However, the prosthesis 30 incorrectly identifies stairs and switches into the corresponding stair mode, which is transferred to the augmented-reality device as status-related data of the prosthesis 30. The said augmented-reality device indicates the stair mode in the form of virtual stairs 310, whereby the orthopedic technician immediately identifies that the prosthesis 30 is in the wrong mode. This facilitates the error search.

Finally, FIG. 6 shows an example in which a movement sequence of a patient 200 is superimposed. In this case, a further additional movement sequence 200′ is superimposed into the field of vision of the orthopedic technician, the additional movement sequence for example having been recorded previously as a virtual twin or having been generated artificially from appropriate data. Thus, the technician can perform a comparison and comprehend the effects of the most recent changes to the prosthesis. In this case, there can be a comparison with previously recorded data from the same patient, a display of physiologically ideal data and/or a representation of a simulation of the expected movement.

In this case, the augmented-reality device can be assisted by an additional camera 14 as an external sensor.

Moreover, the augmented reality is displayed by a tablet 15 in the exemplary embodiment of FIG. 6. In this case, the camera of the tablet 15 is directed at the patient 200 and records their movements. The movement of the patient 200 recorded by the camera of the tablet 15 is then immediately displayed on the display and augmented by the appropriate information.

### LIST OF REFERENCE SIGNS

10 Augmented-reality device
11 Computing unit/evaluation unit
12 Projection device/glasses
13 Lenses of the glasses
14 External camera
15 Projection device/tablet
20 Data/information
20 Data/information visible in the field of vision
21′ Status information
22′ Force vector
23′ Knee angle
24′ Set down points
30 Prosthesis
31 Prosthetic foot
100 Field of vision
200 Patient
200′ Superimposed movement sequence
300 Ramp
310 Stairs

1. A method of computer-based configuration of an orthopedic apparatus worn on the body of a patient (200) equipped therewith, wherein the method comprises the following steps:
acquiring status-related and/or movement-related data (20, 20′) of the orthopedic apparatus during a movement sequence (200′), within the scope of which the relevant patient (200) carries out a movement using the orthopedic apparatus,
transferring the acquired status-related and/or movement-related data (20, 20′) regarding the movement sequence (200′) to an augmented-reality device (10) used by a user, and
superimposing, in the form of augmented reality, at least some of the transferred status-related and/or movement-related data (20, 20′) and/or information (20, 20′, 21′) derived therefrom into the reality perceived by the user using the augmented-reality device (10).

2. The method as claimed in claim 1, characterized in that at least some of the transferred status-related and/or movement-related data (20, 20′) and/or information (20, 20′, 21′) derived therefrom are superimposed, in the form of augmented reality, during the movement sequence (200′) into the reality of the movement sequence (200′) perceived by the user using the augmented-reality device (10).

3. The method as claimed in claim 1 or 2, characterized in that a camera (14) connected to the augmented-reality device (10) is used to record the movement sequence (200′) of the movement performed by the relevant patient (200) with the orthopedic apparatus, and at least one positionally accurate representation position is ascertained on the basis of the recorded movement sequence (200′), the data (20, 20′) and/or information (20, 20′) correlating with the said representation position, wherein the transferred status-related and/or movement-related data (20, 20′) and/or the information (20, 20′, 21′) derived therefrom are superimposed, in the form of augmented reality and in positionally accurate fashion, into the reality perceived by the user at the ascertained position.

4. The method as claimed in any of the preceding claims, characterized in that a movement sequence (200′) stored in a data memory and recorded previously, or a part thereof, is provided, wherein the augmented-reality device (10) is used to superimpose, in the form of augmented reality, the stored and previously recorded movement sequence (200′), or a part thereof, into the reality perceived by the user.

5. The method as claimed in any of claims 1 to 4, characterized in that the data (20, 20′) and/or information (20, 20′) are presented on a display device worn in front of the eyes of the user, in such a way that the data (20, 20′) and/or information (20, 20′) are superimposed, in the form of augmented reality, into the reality perceived by the user.

6. The method as claimed in any of claims 1 to 4, characterized in that a camera (14) connected to the augmented-reality device (10) is used to record the movement sequence (200′) of the movement performed by the relevant patient (200) with the orthopedic apparatus and play back the said movement sequence in real time on a display of the augmented-reality device (10), wherein the data (20, 20′) and/or information (20, 20′) in the representation of the recorded movement sequence (200′) played back on the display are superimposed, in the form of augmented reality, into the reality perceived by the user.

7. The method as claimed in any of the preceding claims, characterized in that the status-related and/or movement-related data (20, 20′) are acquired from a sensor source external to the orthopedic apparatus, from a sensor source integrated in the orthopedic apparatus and/or from a patient-related sensor source secured to the patient (200).

8. The method as claimed in any of the preceding claims, characterized in that a camera (14) connected to the augmented reality device (10) is used to record the movement sequence (200′) of the movement performed by the relevant patient (200) with the orthopedic apparatus, and an evaluation unit (11) is used to ascertain movement-related data (20, 20′) and/or information (20, 20′) derived therefrom from the recorded movement sequence (200′) by way of image recognition carried out by the evaluation unit (11), the said movement-related data and/or information derived therefrom then being superimposed in the form of augmented reality.

9. The method as claimed in any of the preceding claims, characterized in that setting parameters of the orthopedic apparatus, as status-related data (20, 20′) and/or information (20, 20′, 21′) derived therefrom, are transferred to the augmented-reality device (10) or are already stored therein, and the said setting parameters are superimposed, in the form of augmented reality, into the reality perceived by the user.

10. The method as claimed in any of the preceding claims, characterized in that targets in relation to status-related and/or movement-related data (20, 20′) and/or information (20, 20′, 21′) derived therefrom are provided in a database, wherein at least some of the targets are read from the database and are superimposed, in the form of augmented reality, into the reality perceived by the user, directly and/or as deviations of the status-related and/or movement-related data from the targets.

11. The method as claimed in any of the preceding claims, characterized in that a target movement sequence is provided in a database, wherein at least some of the target movement sequence is read from the database and superimposed, in the form of augmented reality, into the reality perceived by the user.

12. The method as claimed in claim 11, characterized in that deviations from the target movement sequence are determined on the basis of the status-related and/or movement-related data (20, 20′) and/or information (20, 20′, 21′) derived therefrom using an evaluation unit (11) and are superimposed, in the form of augmented reality, into the reality perceived by the user.

13. The method as claimed in any of the preceding claims, characterized in that a camera (14) connected to the augmented-reality device (10) is used to record the movement sequence (200′) of the movement performed by the relevant patient (200) with the orthopedic apparatus, and, by means of an evaluation unit (11), a virtual twin is created and stored in a data memory.

14. The method as claimed in claim 13, characterized in that recording the movement sequence (200′) is followed by superimposing the virtual twin, in the form of augmented reality, into the field of vision (100) of the user.

15. The method as claimed in claim 13, characterized in that a modified movement sequence (200′) of the virtual twin is calculated on the basis of a change in the settings of the orthopedic apparatus and/or by a simulation of external influences on the movement sequence (200′) by means of a computing unit (11), wherein the modified movement sequence (200′) is superimposed, in the form of augmented reality, into the field of vision (100) of the user.

16. The method as claimed in any of the preceding claims, characterized in that a deviation between the acquired status-related and/or movement-related data (20, 20′) of the orthopedic apparatus and the information (20, 20′, 21′) derived from the virtual twin is ascertained by means of the evaluation unit (11) and is superimposed, in the form of augmented reality, into the field of vision (100) of the user.

17. A system for configuring an orthopedic device, configured to carry out the method as claimed in any of the preceding claims.
